(19) United States
(12) Patent Application Publication
(10) Pub. No.: US 2023/0127564 A1
(43) Pub. Date: Apr. 27, 2023

(54) PROCESSES FOR REMOVING AND/OR REDUCING PERMANGANATE REDUCING COMPOUNDS AND ALKYL IODIDES

(71) Applicant: Celanese International Corporation, Irving, TX (US)

(72) Inventors: Himanshu LODHA, Houston, TX (US); Ronald D. SHAVER, Houston, TX (US)

(73) Assignee: Celanese International Corporation, Irving, TX (US)

(21) Appl. No.: 17/911,352

(22) PCT Filed: Mar. 26, 2021
(86) PCT No.: PCT/US2021/024320

§ 371 Date: Sep. 13, 2022

Related U.S. Application Data
(60) Provisional application No. 63/003,681, filed on Apr. 1, 2020.

(51) Int. Cl. C07C 51/44; C07C 51/12

(52) U.S. Cl. CPC C07C 51/44; C07C 51/12

(57) ABSTRACT

Continuous acetic acid production with a process to remove and/or reduce permanganate reducing compounds (PRCs), including acetaldehyde. The process involves obtaining a stream comprising the PRCs and removing water from the stream through a dehydrating step, involving distillation, extraction, or phase separation. After removing water, an alkane may be combined to enhance the separation of PRCs from alkyl iodides.

### PRIORITY

This application claims priority to U.S. Provisional Application No. 63/003,681, filed on Apr. 1, 2020, which is incorporated herein by reference.

### FIELD OF THE INVENTION

This invention relates generally to processes for continuous acetic acid production, in particular, to improved processes for removing and/or reducing permanganate reducing compounds, such as acetaldehyde, and alkyl iodides, such as methyl iodide.

### BACKGROUND OF THE INVENTION

Methanol carbonylation is an industrially successful process for obtaining acetic acid in the presence of a reaction mixture containing homogeneous catalyst. Despite the high yields of acetic acid, the process is known to generate impurities resulting in low purity acetic acid. One such impurity that has received considerable attention is acetaldehyde because of the relevant difficulty in removal, acetaldehyde is a precursor to several other impurities, and the impact on purity of acetic acid. For example acetaldehyde has a close boiling point to an effective catalyst promoter, which makes simple distillation insufficient. To overcome these insufficiencies, several proposals have been to remove acetaldehyde by alkane or water extraction, or by reaction with amino compounds, oxygen-containing gases, and hydroxyl compounds. Unfortunately, despite the use of these treatments, acetaldehyde continues to be challenge in obtaining high purity acetic acid. Further, formation of acetaldehyde derived impurities reduces the efficiency when removing acetaldehyde.

U.S. Pat. No. 5,625,095 discloses a high purity acetic acid is prepared by reacting methanol with carbon monoxide in the presence of a rhodium catalyst, iodide salts, and methyl iodide, wherein an acetaldehyde concentration in the reaction liquid is maintained at 400 ppm or lower. This may be attained by contacting the liquid containing carbonyl impurities with water to separate and remove the carbonyl impurities. After that, the liquid can be returned to the reactor.

U.S. Pat. No. 6,143,930 discloses a method to manufacture high purity acetic acid. This process is described in relation to that produced by a low water carbonylation process but is applicable to other mechanisms for production of acetic acid which results in formation of permanganate reducing compounds such as acetaldehyde, propionic acid, and alkyl iodide impurities in intermediate process streams. It has been found that permanganate reducing compounds and alkyl iodides may be conveniently removed from a light phase of an intermediate stream in the reaction process by employing a multiple distillation process coupled with an optional extraction of acetaldehyde. The distillation process involves first distilling a light phase to concentrate the permanganate reducing compounds, and in particular the acetaldehyde, and then separating the permanganate reducing compounds and alkyl iodides in a second distillation tower. The second distillation serves to remove the permanganate reducing compounds and alkyl iodides from methyl iodide, methyl acetate, and methanol mixture. As an optional third step, the twice distilled stream may be directed to an extractor to remove any remaining quantities of methyl iodide from the aqueous acetaldehyde stream to obtain acetic acid as a final product in greater than 99% purity.

U.S. Pat. No. 9,862,669 describes processes for producing acetic acid and, in particular, to improved processes for recovering C₂+ alkyl halides and removing permanganate reducing compounds formed during the carbonylation of methanol in the presence of a Group VIII metal carbonylation catalyst to produce acetic acid.

U.S. Pat. No. 10,562,836 describes a process for separating or removing permanganate reducing compounds (PRCs) from a first mixture containing at least one PRC, methyl iodide, and water comprises the steps of: feeding the first mixture to a feed port of a distillation column, and distilling and separating the first mixture into an upper stream and a lower stream, wherein the distillation of the first mixture forms a second mixture at an upper position than the feed port, and the process further comprises the steps of: withdrawing the second mixture as the upper stream, and withdrawing the lower stream from a lower position than the feed port.

U.S. Pat. No. 10,562,836 describes a process for producing acetic acid while efficiently separating permanganate reducing compounds (PRCs) and methyl iodide is provided. PRCs are separated or removed from a mixed composition containing PRCs and methyl iodide by distilling the mixed composition in a distillation step to form an overhead stream, a side-cut stream (5B), and a lower stream. In a distillation column of the distillation step, an extractant (e.g., water) extracting PRCs preferentially to methyl iodide is added to a concentration zone in which PRCs and methyl iodide are concentrated, and an extraction mixture falling from the concentration zone is withdrawn as the side-cut stream.

Although existing carbonylation processes are highly efficient, further improvements for the recovery of acetic acid, with a reduction in impurities, in a safe and efficient manner is needed.

### SUMMARY OF THE INVENTION

This invention generally relates to processes for removing and/or reducing permanganate reducing compounds (PRCs) and alkyl iodides by removing water followed by contacting with an alkane, and in particular butane or pentane. For purposes of the present disclosure PRCs may include acetaldehyde, acetone, methyl ethyl ketone, butylaldehyde, crotonaldehyde, 2-ethyl crotonaldehyde, 2-ethyl butyraldehyde, and the aldol condensation products thereof. PRCs, in particular acetaldehyde, deteriorate the quality of the acetic acid, and thus reducing and/or removing the PRCs may improve the acetic acid product. Despite the need to remove PRCs, there are challenges to effectively separate PRCs without a loss of methyl iodide. The use of the alkanes described herein provides a solution that can provide improvements to the quality of the acetic acid product.

In one embodiment there is a provided a method for removing permanganate reducing compounds (PRCs) formed in the production of acetic acid, comprising reacting methanol and carbon monoxide in a suitable liquid phase reaction medium comprising a Group VIII metal catalyst, an alkyl iodide, and water; separating the liquid phase reaction medium into a vapor stream comprising acetic acid and at least one PRCs, and a residuum catalyst stream; distilling at least a portion of the vapor stream (e.g., either a light or heavy phase) in a first distillation column to yield a side stream comprising acetic acid and a first overhead comprising alkyl iodide, water, and PRCs; removing water from at least a portion of the first overhead to obtain a process stream and combining at least a portion of the process stream with at least one alkane, such as butane, to obtain a combined stream having a reduce amount of water. The process further comprises distilling the combined stream to yield a second overhead comprising the at least one alkane and PRCs, and a lower stream comprising the alkyl iodide; and biphasically separating the second overhead to remove PRCs in an aqueous stream, wherein a stream comprising water is introduced to the vessel.

### BRIEF DESCRIPTION OF THE DRAWINGS

The present invention will be better understood in view of the appended non-limiting figures, wherein:

FIG. 1 illustrates a process for producing acetic acid with PRCs removal in accordance with embodiments of the present invention.

FIG. 2 illustrates a process for removing PRCs from a light phase in accordance with embodiments of the present invention.

FIG. 3 illustrates a process for removing PRCs from a heavy phase in accordance with embodiments of the present invention.

### DETAILED DESCRIPTION OF THE INVENTION

In general, the present invention is useful for continuous acetic acid production to obtain a high quality acetic acid product. This process improves the removal and/or reduction of impurities such as PRCs, in particular acetaldehyde, without having to decrease production rates. In addition, the process may provide improvements to the retention of valuable catalyst components, including alkyl iodides, while removing and/or reducing PRCs.

### Carbonylation Reaction

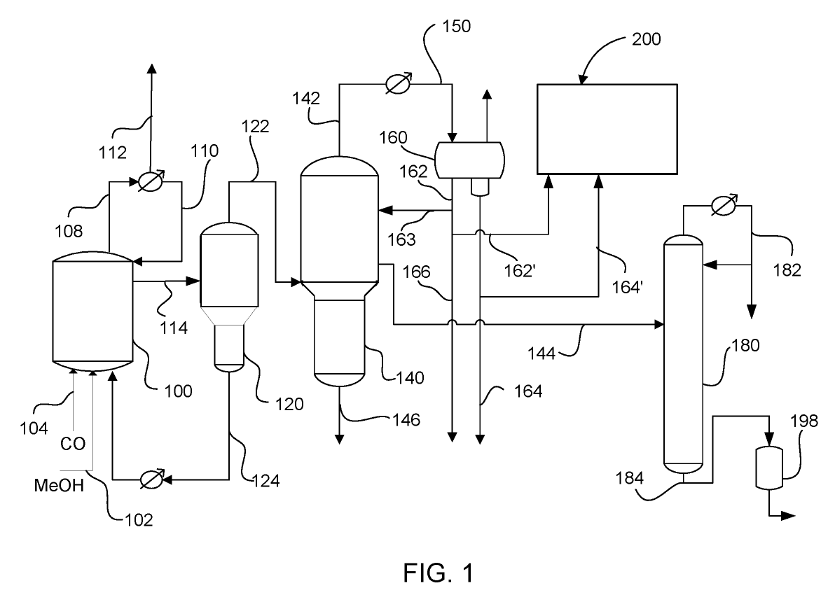

The process in FIG. 1 may be used for the production of acetic acid by carbonylation of methanol. Although a process is shown in FIG. 1, those skilled in the art will understand that there are several processes and variations that may be employed with the improvements described herein. As described further below, the apparatuses used with the process and variations may include a reactor vessel, flash vessel, one or more distillation columns, decanters, ion exchange resin beds, absorption systems, and/or extractors, along with the associated heat exchanges, pumps, condensers, receivers, reboilers, valves, pipes and process controllers. Each of the vessels and column may include various internals that are known to those in skilled in the art.

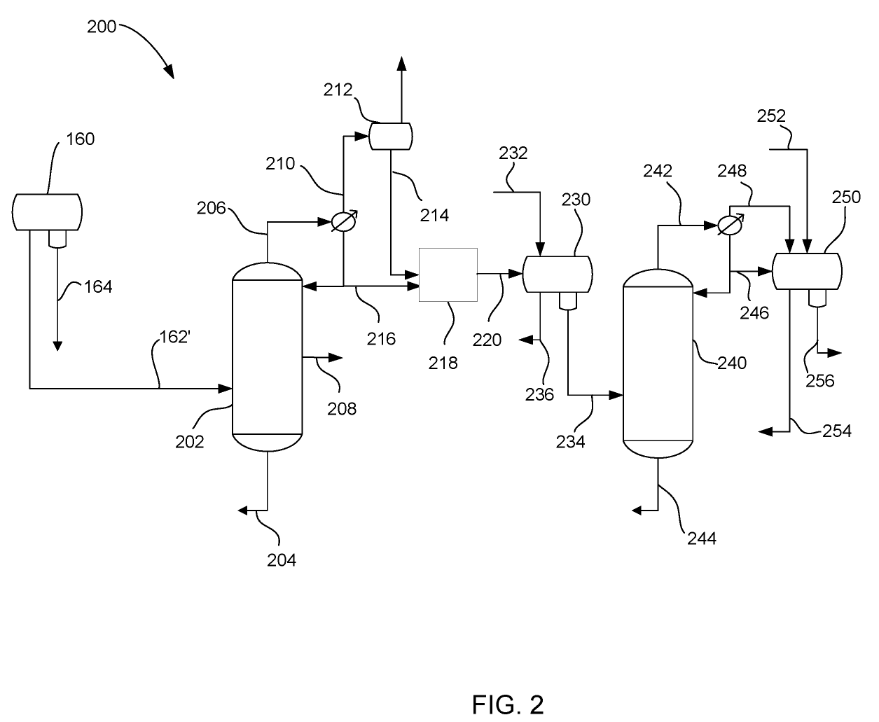

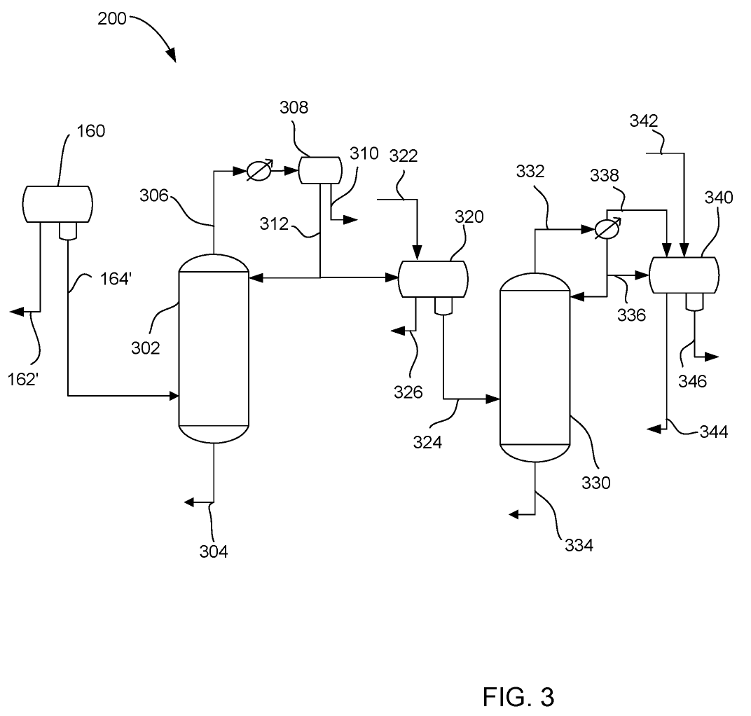

FIG. 1 is a schematic carbonylation process that is sufficient for continuously producing acetic acid, while reducing impurities. This process comprises a reactor 100, a flash vessel 120, distillation columns 140, and 180, and process for removing and/or reducing PRCs shown by 200 and described in further detail in FIGS. 2 and 3.

The reactor 100 is a unit for performing the homogenous carbonylation reaction step in the liquid phase. The reaction step is for carbonylating methanol to produce acetic acid in a continuous manner. The starting materials in the reaction are liquid methanol and gaseous carbon monoxide. As shown, methanol-containing feed stream 102 and carbon monoxide-containing feed stream 104 are directed to reactor 100, in which the carbonylation reaction occurs to form acetic acid. Although not shown, a flow transmitter may be present on the both feed streams to control and/or monitor the flow of each respective stream.

Methanol-containing feed stream 102 may comprise at least one member selected from the group consisting of methanol, dimethyl ether, and methyl acetate. In one embodiment, methanol-containing feed stream 102 comprises methanol in a concentration of at least 60 wt.% to up to and including 100 wt.%. Methanol-containing feed stream 102 may be derived in part from a fresh feed from a reservoir tank (not shown), a recycled feed from the system, or a combination of fresh and recycles feeds. At least some of the methanol and/or reactive derivatives thereof will be converted to, and hence present as, methyl acetate in the liquid medium by esterification reaction with acetic acid.

Carbon monoxide-containing feed stream 104 may comprise primarily carbon monoxide of greater than or equal to 95 vol.%, e.g., greater than or equal to 97 vol.% or greater than or equal to 99 vol.%. In some embodiments, minor impurities such as hydrogen, carbon dioxide, oxygen, and/or nitrogen may be present in amount of less than 5 vol.%, e.g., less than 3 vol.% or less than 1 vol.%. These minor impurities may also be generated by various side reactions under operating conditions.

The carbon monoxide partial pressure, at an absolute pressure, in the reactor may vary widely but is typically from 2 to 30 atm (absolute pressure), e.g., from 3 to 18 atm or from 6 to 15 atm. Hydrogen, which may be generated in the reaction or may be supplied as needed, increases the catalytic activity but can also result in formation of byproducts, including acetaldehyde. The hydrogen partial pressure, at an absolute pressure, in the reactor is typically from 0.05 to 5 atm, e.g., from 0.25 to 2 atm or from 0.3 to 1.8 atm. The total reactor internal pressure may range from 15 to 40 atm (absolute pressure). In some embodiments, the internal pressure of the reactor 100 may be controlled by withdrawing a purge stream 108, which is a gaseous stream. In some embodiments, the internal pressure may be constant or substantially constant, which refers to minor variations in pressure over a continuous process that does not impact production rates. By carrying out the reaction under such pressure conditions, acetic acid is efficiently produced while inhibiting various byproducts and side reactions.

Typical carbonylation reaction temperatures may be greater than or equal to 150° C., e.g., greater than or equal to 175° C. or greater than or equal to 185° C. In terms of ranges, the carbonylation reaction temperature may be from 150° C. to 250° C., e.g., from 175° C. to 230° C. or from 185° C. to 205° C. The carbonylation reaction is exothermic and temperature of the reactor may be regulated by a variety of methods. For purposes of the present disclosure, any suitable cooling may be used to regulate the temperature of the reactor. U.S. Pat. No. 5,374,774 describes a cooling unit in the recycle line for the reactor. A pump around loop may be used to generate additional heat for the production of steam while regulating the temperature of the carbonylation reactor, which is further described in U.S. Pat. No. 8,530,696. In some embodiments, the temperature of the reactor may be controlled by condensing a portion of the flash overhead that is returned to the reactor, which is further described in U.S. Pat. No. 8,957,248.

The production rate of acetic acid may be from 5 to 50 mol/L·h, e.g., from 10 to 40 mol/L·h, or from 15 to 35 mol/L·h. “Greater production rates” generally refers to operating above 20 mol/L·h.

Carbon monoxide is introduced at a rate sufficient to maintain the desired internal reactor pressure. In some embodiments, carbon monoxide is continuously introduced through stream 104 into the carbonylation reactor 100, desirably below the agitator, which may be used to stir the contents, and thoroughly disperse the carbon monoxide throughout the liquid reaction medium. Other methods of agitating the reaction medium may be employed, such as a vessel with an eductor or pump-around mixing, or bubble-column type vessel, with or without an agitator.

The material of the carbonylation reactor and its internals is not particularly limited and may be a metal, a ceramic, a glass, or combinations thereof. For example, the material may include zirconium-based materials and alloys that tend to have high corrosion resistance, but may also include iron-based alloys (stainless steel), nickel-base alloys (HASTELLOY™ or INCONEL™), titanium-based materials and alloys, or aluminum-based materials or alloys.

Under continuous production conditions, various gas-phase components may be formed or evolved from the liquid reaction. The gas-phase component can include carbon monoxide, hydrogen, methane, carbon dioxide, acetic acid, methyl acetate, methyl iodide, hydrogen iodide, acetaldehyde, dimethyl ether, and water. In some embodiments, the purge stream 108 contains low amounts of hydrogen iodide of less than or equal to 1 vol.%, e.g., less than or equal to 0.9 vol.%, less than or equal to 0.8 vol.%, less than or equal to 0.7 vol.%, or less than or equal to 0.5 vol.%. To prevent an undesirable buildup of various gas-phase components, a purge stream 108 is drawn from the upper portion of the reactor 100.

Venting purge stream 108 from the reactor 100 further reduces the buildup of gaseous byproducts and maintains a set carbon monoxide partial pressure at a given total reactor pressure. To prevent loss of useful components, the purge stream 108 may be cooled by heat exchange with a cooling medium in one or more condensers to partially condense any condensable liquids present as vapors in the gaseous purge stream into a condensate portion 110 and a gaseous portion 112. Condensate portion 110 typically includes useful liquid products, such as acetic acid, methyl acetate, methyl iodide, acetaldehyde, dimethyl ether, and water, and is returned to the reactor 100. Although the gaseous portion may be flared, the gaseous portion 112 typically includes sufficient amounts of carbon monoxide, hydrogen, methane, carbon dioxide and minor amounts of iodides such as methyl iodide or hydrogen iodide to make further recovery desirable.

The gaseous portion 112 (portion of the first gaseous stream) may be further processed in an absorption system (not shown), such as a scrubber system or a pressure swing absorption tower. Such absorption systems are widely known to those skilled in the art, and are described in U.S. Pat. Nos. 4,241,219, 8,318,977, 9,233,907, and EP2637767B1, the entire contents and disclosure of which is hereby incorporated by reference.

Returning to the reactor 100, the catalyst in the reaction medium plays the role of promoting the methanol carbonylation reaction. In commercial production, the Group VIII metal catalyst does not activate methanol directly, so a more reactive methyl substrate (reactant) must be generated in situ. An iodide promoter, such as hydrogen iodide, converts the methanol into methyl iodide. However, since most of the reaction medium is acetic acid, the methanol is esterified to methyl acetate, which is activated by hydrogen iodide into methyl iodide.

The components of the reaction medium are maintained within defined limits to ensure sufficient production of acetic acid and utilization of reactants, while limiting the production of byproducts. The following amounts are based on the total weight of the liquid phase of the reaction medium. In a continuous process, the amounts of components are maintained within the ranges provided and fluctuations within these ranges are anticipated. One of ordinary skill would readily understand how to control the process to maintain the amounts of components in the reaction medium.

The reaction medium includes a concentration of the Group VIII metal catalyst, e.g., cobalt, rhodium, iridium, or combinations thereof, in an amount from 200 to 3000 wppm based on the metal in the reaction medium, e.g., from 800 to 3000 wppm, or from 900 to 1500 wppm.

Water in the reaction medium is a useful component for forming acetic acid according to the methanol carbonylation reaction mechanism, and further dissolves soluble components in the reaction medium. The concentration of water in the reaction medium is maintained to be less than or equal to 14 wt.%, e.g., from 0.1 wt.% to 14 wt.%, from 0.2 wt.% to 10 wt.% or from 0.25 wt.% to 5 wt.%. To control the water concentration, water may be continuously fed to the carbonylation reactor 100, including through the recycles lines, at a predetermined flow rate. In some embodiments, the reaction is conducted under low water conditions and the reaction medium contains water in an amount from 0.1 to 4.1 wt.%, e.g., from 0.1 to 3.1 wt.% or from 0.5 to 2.8 wt.%. In another embodiment, the reaction is conducted with water in an amount of less than or equal to 2 wt.% water, e.g., from 0.1 to 2 wt.%, or from 0.1 to 1.9 wt.%.

The promoter in the reaction medium may be an iodide to assist the activity of the catalyst. Non-limiting examples of the iodide as the promoter include methyl iodide, an ionic iodide, and combinations thereof. The concentration of methyl iodide in the reaction medium is maintained to be from 1 to 25 wt.%, e.g., from 5 to 20 wt.%, or from 4 to 13.9 wt.%. The ionic iodide can stabilize the metal catalyst and inhibit side reactions. Non-limiting examples of the ionic iodide include lithium iodide, sodium iodide, and potassium iodide. The concentration of iodide salt, e.g., lithium iodide, in the reaction medium is maintained to be from 1 to 25 wt.%, e.g., from 2 to 20 wt.%, or from 3 to 20 wt.%. The iodide salt may be formed in situ, for example, by adding lithium acetate, lithium carbonate, lithium hydroxide or other lithium salts of anions compatible with the reaction medium. In some embodiments, the process may maintain a concentration of lithium acetate in the reaction medium from 0.3 to 0.7 wt.%, e.g., from 0.3 to 0.6 wt.%.

It will be generally recognized that it is the concentration of iodide ion in the catalyst system that is important and not the cation associated with the iodide, and that at a given molar concentration of iodide, the nature of the cation is not as significant as the effect of the iodide concentration. Any metal iodide salt, or any iodide salt of any organic cation, or other cations such as those based on amine or phosphine compounds (optionally, ternary or quaternary cations), can be maintained in the reaction medium provided that the salt is sufficiently soluble in the reaction medium to provide the desired level of the iodide. When the iodide is a metal salt, preferably it is an iodide salt of a member of the group consisting of the metals of Group IA and Group IIA of the periodic table as set forth in the “Handbook of Chemistry and Physics” published by CRC Press, Cleveland, Ohio, 2002-03 (83rd edition). In particular, alkali metal iodides are useful, with lithium iodide being particularly suitable.

As described above, the methyl acetate may be formed by the reaction between acetic acid and methanol. The concentration of methyl acetate in the reaction medium is maintained to be from 0.5 to 30 wt.%, e.g., from 0.5 to 20 wt.%, from 0.6 to 9 wt.%, or from 0.6 to 4.1 wt.%.

Acetic acid is the main product of the reaction and the concentration of acetic acid in the reaction medium, which also functions as solvent, is generally in amount of greater than or equal to 30 wt.%, e.g., greater than or equal to 40 wt.% or greater than or equal to 50 wt.%. The acetic acid in the reaction medium includes acetic acid previously charged into the reactor upon start-up.

In addition to the acetic acid product, various byproducts may also be generated in the reaction medium. Non-limiting examples of the byproducts also include hydrogen, methane, carbon dioxide, formic acid, hydrogen iodide, acetic anhydride, acetaldehyde, crotonaldehyde, 2-ethyl crotonaldehyde, dimethyl ether, propionic acid, and alkyl iodides such as ethyl iodide, hexyl iodide, and decyl iodide. Hydrogen iodide is formed via the reaction mechanism of the methanol carbonylation reaction when the catalyst alone or in combination with the promoter as described above is used. The reaction medium may have an acetaldehyde concentration ranging from 0 to 1800 wppm of the total reaction medium, e.g., from 200 to 1600 wppm, or from 350 to 1000 wppm. The reaction medium may have a hydrogen iodide concentration ranging from 50 to 5000 wppm of the total reaction medium, e.g., from 100 to 3000 wppm, or from 200 to 2000 wppm. In some embodiments, the reaction medium may also include acetic anhydride. The reaction medium may have an acetic anhydride concentration ranging from 0 to 5000 wppm of the total reaction medium, e.g., from 0.01 to 3000 wppm, or from 0.1 to 1000 wppm.

Byproducts may be controlled by regulating the reaction medium and, in addition, the byproducts may be removed by separation process as described further herein. For example, as described in U.S. Pat. No. 8,017,802, formic acid may be controlled by the water content in the reactor resulting in a formic acid content in the acetic acid product that is less than 160 wppm, e.g., less than 140 wppm, or less than 100 wppm. Separation of byproducts may be limited by the associated costs. When the byproducts are not removed, especially higher boiling point components, the components can concentrate in the acetic acid product. Thus, it is useful to limit the production of byproducts in the reactor to reduce the need for separation. For example, in some embodiments, the propionic acid concentration in the acetic acid product may further be maintained below 250 wppm by maintaining the ethyl iodide concentration in the reaction medium at less than or equal to 750 wppm, e.g., less than or equal to 350 wppm, without removing propionic acid from the acetic acid product.

The various ranges for the maintaining the components for higher production rates are as follows. In some embodiments, the production rate of acetic acid is from 15 mol/L·h to 35 mol/L·h, the concentration of the rhodium catalyst in the reaction medium is maintained from 900 to 1500 wppm, the concentration of water is maintained from 0.5 to 2.8 wt.%, the concentration of methyl iodide is maintained from 4 to 13 wt.%, the concentration of lithium iodide is maintained from 3 to 20 wt.%, the concentration of lithium acetate is maintained from 0.35 to 0.55 wt.%, the concentration of methyl acetate is maintained from 2 to 3.5 wt.%, and the concentration of hydrogen iodide is maintained at less than or equal to 0.5 wt.%. The balance of the reaction medium is acetic acid.

### Flash Vessel

In steady state operations, the reaction medium is continuously withdrawn from the reactor 100 through stream 114 at a rate sufficient to maintain a constant level therein. For purposes of the present invention the flash flow or second mass flow rate refers to the withdrawn reaction medium from the reactor. To obtain the acetic acid product, the withdrawn reaction medium in stream 114 is fed to the subsequent downstream flash vessel 120, which is also referred to as an evaporator. In some embodiments, a converter reactor (not shown) or a pipe reactor (not shown) can be employed between the reactor and flash vessel. A pipe reactor is described in U.S. Pat. No. 5,672,744 and is used to react the dissolved carbon monoxide in the reaction medium. Chinese Patent No. CN1043525C describes a converter reactor to allow the reaction to proceed to a greater extent prior to subsequent flashing. The converter reactor produces a vent stream comprising gaseous components which are typically scrubbed with a compatible solvent to recover components such as methyl iodide and methyl acetate.

To cause separation of the acetic acid from the catalyst, the reaction medium is depressurized and flash vessel 120 is controlled to maintain a pressure considerably lower than the pressure in the carbonylation reactor. Those skilled in the thermodynamic arts are aware that this pressure reduction is accompanied by flashing of the more volatile components of the liquid reaction product solution. In this way, the carbonylation product, which has a higher vapor pressure than the catalyst, is removed as a vapor leaving behind the catalytic agent in the unflashed liquid carbonylation product solution. This separation is desirable to obtain a fraction of the product as a vapor and is separated from the Group VIII metal catalyst which remains in the liquid phase. Thus, the high cost catalyst may be recycled back into the reactor, while the vapor product is provided for further purification without entrainment from the Group VIII metal catalyst.

Flash vessel 120 performs a flash evaporation or distillation step, referred to herein as flashing or evaporating, to return the residuum catalyst stream 124 to the reactor 100 and separate a vapor product stream 122 comprising acetic acid for further processing. In some embodiments, the flashing may be performed by decompressing the reaction medium in stream 114 with or without heating. Stream 114 may be tangentially fed through one or more feed ports as shown in U.S. Pat. No. 6,599,348. To direct the liquid portion downwards, a splash plate may be used in each of the feed ports.

Although the flash vessel 120 is illustrated has having a tapered base design, those of ordinary skill in the art will recognize that various cylindrical designs having other geometrical configurations may be used.

In some embodiments, the flashing may be carried without or with heating. An adiabatic flashing is generally employed. The temperature of the flash vessel 120 may be from 80° C. to 260° C., e.g., 100° C. to 200° C. The internal pressure (gauge) of the flash vessel 120 is typically from 0.5 atm to 5 atm, e.g., from 0.5 atm to 3.5 atm, 0.5 to 2.5 atm, or from 0.5 to 1.5 atm.

The mass ratio of the vapor product stream 122 to the residuum catalyst stream 124, which are separated from each other, may be from 10:90 to 50:50, e.g., from 20:80 to 40:60. The vapor product stream 122 comprises acetic acid, as well as methyl iodide, methyl acetate, water, PRCs, and other byproducts or impurities. Dissolved gases in the reaction medium that enter the flash vessel are concentrated into stream 122. The dissolved gases comprise a portion of the carbon monoxide and may also contain gaseous byproducts such as methane, hydrogen, and carbon dioxide. In some embodiments, a mist eliminator or similar plate may be employed near the vapor outlet to coalesce liquid droplets. An optional scrubbing section (not shown) may further be employed in the vapor outlet of the flash vessel to reduce entrainment from metallic catalysts or other metallic components into the vapor stream. A wash liquid may be introduced into the optional scrubbing section.

Vapor product stream 122 comprises acetic acid, methyl iodide, methyl acetate, water, acetaldehyde, and hydrogen iodide. The flash vessel 120 may be operated under conditions sufficient to vaporize at least 80% of the methyl iodide and methyl acetate, based on the total reaction medium, into the vapor product stream 122. In some embodiments, vapor product stream 122 comprises acetic acid in an amount from 45 to 75 wt.%, methyl iodide in an amount from 20 to 50 wt.%, methyl acetate in an amount of less than or equal to 9 wt.%, and water in an amount of less than or equal to 15 wt.%, based on the total weight of the vapor product stream. In another embodiment, vapor product stream 122 comprises acetic acid in an amount from 45 to 75 wt.%, methyl iodide in an amount from 24 to less than or equal to 36 wt.%, methyl acetate in an amount of less than or equal to 9 wt.%, and water in an amount of less than or equal to 15 wt.%, based on the total weight of the vapor product stream. More preferably, vapor product stream 122 comprises acetic acid in an amount from 55 to 75 wt.%, methyl iodide in an amount from 24 to 35 wt.%, methyl acetate in an amount from 0.5 to 8 wt.%, and water in an amount from 0.5 to 14 wt.%. In yet a further preferred embodiment, vapor product stream 122 comprises acetic acid in an amount from 60 to 70 wt.%, methyl iodide in an amount from 25 to 35 wt.%, methyl acetate in an amount from 0.5 to 6.5 wt.%, and water in an amount from 1 to 8 wt.%. The acetaldehyde concentration in the vapor product stream 122 may be in an amount from 0.005 to 1 wt.%, based on the total weight of the vapor product, e.g., from 0.01 to 0.8 wt.%, or from 0.01 to 0.7 wt.%. Vapor product stream 122 may comprise hydrogen iodide in an amount less than or equal to 1 wt.%, based on the total weight of the vapor product stream, e.g., less than or equal to 0.5 wt.%, or less than or equal to 0.1 wt.%. The propionic acid, acetic anhydride, or formic acid, if present, may be present in amounts in vapor product stream 122 in a reduced amount of less than 1 wt.%, e.g., less than 0.5 wt.%.

In some embodiments, the entire vapor product stream 122 is directed to the light ends column 140 as a vapor stream. This provides the heat to separate the components in the light ends column 140. Alternatively, there may be provided by some embodiments a condenser that cools and partially condenses a portion of the vapor product stream 122.

To handle the residuum catalyst stream 124 in a manner that maintains flow rates, prevents equipment damage, and provides sufficient control, a vortex breaker (not shown) may be used near the liquid outlet of the flash vessel 120. Residuum catalyst stream 124 comprises acetic acid, the metal catalyst, corrosion metals, as well as other compounds that remain without volatilization in the flashing step. In some embodiments, liquid recycle stream comprises acetic acid in an amount from 60 to 90 wt.%, metal catalyst in an amount from 0.01 to 0.5 wt.%, corrosion metals (e.g., nickel, iron and chromium) in a total amount from 10 to 2500 wppm, lithium iodide in an amount from 5 to 20 wt.%, methyl iodide in an amount from 0.5 to 5 wt.%, methyl acetate in an amount from 0.1 to 5 wt.%, water in an amount from 0.1 to 8 wt.%, acetaldehyde in an amount of less than or equal to 1 wt.% (e.g., from 0.0001 to 1 wt.% acetaldehyde), and hydrogen iodide in an amount of less than or equal to 0.5 wt.% (e.g., from 0.0001 to 0.5 wt.% hydrogen iodide).

The residuum catalyst stream 124 may be predominantly acetic acid containing the rhodium and the iodide salt along with lesser quantities of methyl acetate, methyl iodide, lithium acetate and water and is recycled to the reactor, as discussed above. A suitable pump, such as a centrifugal pump, although not shown is generally employed to return to the residuum catalyst stream 124 to the higher pressure reactor 100. Additional heat exchangers may be used as needed for adjusting the temperature of residuum catalyst stream 124. In one embodiment, prior to returning residuum catalyst stream 124 to the reactor 100, a slip stream may pass through a corrosion metal removal bed, such as an ion exchange bed, to remove any corrosion metals, such as nickel, iron, chromium, and molybdenum, as described in U.S. Pat. No. 5,731,252, which is incorporated herein by reference in its entirety. Corrosion metals if not controlled can lead to decrease in catalytic activity and fouling of the equipment. Also, the corrosion metal removal bed may be used to remove nitrogen compounds, such as amines, as described in U.S. Pat. No. 8,697,908, which is incorporated herein by reference in its entirety.

In some embodiments, to prevent deactivation of the catalyst in the flash vessel, carbon monoxide may be introduced in the lower section of the flash vessel and/or the residuum catalyst stream 124. More specifically, the carbon monoxide is fed into the liquid in the base of the flash vessel that contains the Group VIII metal catalyst. Catalyst deactivation and loss, especially for rhodium catalyst, is generally believed due to carbon monoxide-depleted or low pressure environments in the carbonylation system as are typically experienced in the flash vessel.

The flashing may be performed using a two-stage flash, where the absolute internal pressure in the first flash vessel may be in the range from 1 to 10 atm and the pressure in the second flash vessel may suitably be in the range from 0 to 5 atm. The pressure in both stages operates on the backpressure from the absorption system. In some embodiments, prior to entering the flash vessel 120, the reaction medium may be fed to a pre-flash vessel (not shown) operating at an intermediate pressure between the operating pressures of the carbonylation reactor and a subsequent flash vessel, thereby retaining most of the product acetic acid in solution, while flashing off methyl iodide and methyl acetate. In this embodiment, the methyl iodide and methyl acetate flashed off from the pre-flash vessel may be fed to a condenser or may be sent directly to a low-pressure absorber, thereby reducing the load on a downstream distillation column. Further details are described in U.S. Pat. No. 8,168,822, the entire contents and disclosures of which are hereby incorporated by reference.

### Light Ends Column

As shown in FIG. 1, vapor product stream 122 is continuously introduced into a first column 140, also referred to as a light ends column. Distillation yields a low-boiling overhead stream 142, an acetic acid product that preferably is removed via a side stream 144, and a residue stream 146, which may be recycled to the reactor. In some embodiments, side stream 144 may pass through a side condenser to further reduce impurities. In other embodiments, a vapor portion (not shown) drawn from above the side stream may be condensed to further increase capacity in first column 140 as described in U.S. Pat. No. 7,989,659.

Residue stream 146 contains a larger amount of components having a higher boiling point than that of acetic acid, and contains more of these components than low-boiling overhead stream 142 or side stream 144. In addition, residue stream 146 may also contain entrained catalyst. The components of residue stream 146 also include acetic acid, methanol, methyl acetate, and/or water. Residue stream 146 is recycled to flash vessel 120 or reactor 100. In some embodiments, the continuous process may operate without residue stream 146.

First column 140 may be a plate column, packed column, or a combination thereof. In some embodiments, distillation column 140 is a plate column typically having from 5 to 80 theoretical plates, e.g., from 5 to 50 theoretical plates. The column top pressure (gauge) in first column 140 within the range from 0.5 to 4 atm, e.g., from 0.8 to 3 atm, or from 1 to 2.5 atm. The pressures at the bottom of first column 140 are typically higher and may range from 0.6 to 4.5 atm, e.g., from 0.9 to 3.5 atm, or from 1.1 to 3 atm. In some embodiments, first column 140 operates with a column top temperature that may be controlled at a temperature which is lower than the boiling temperature of acetic acid at the column top pressure, such as from 80° C. to 150° C., e.g., 90° C. to 130° C. The bottom temperature may range from 100° C. to 180° C., e.g., from 120° C. to 165° C. or from 125° C. to 160° C. In some embodiments, the heat for the distillation is primarily provided by vapor product stream 122. In some embodiments, when supplemental heat is required or during start-up, a reboiler may provide further heat for the distillation in first column 140.

In some embodiments, low-boiling overhead stream 142 comprises water, methyl acetate, methyl iodide, hydrogen iodide, acetaldehyde, dimethyl ether, and other light carbonyl impurities, as well as acetic acid. The amount of water in low-boiling overhead stream 142 is generally greater than or equal to 5 wt.%. Low-boiling overhead stream 142 is continuously passed through one or more condensers, which may be supplied with suitable coolant (e.g., cooling water) at a temperature of less than or equal to 60° C., e.g., from 20° C. to 60° C. or from 20° C. to 40° C., to partially condense low-boiling overhead stream 142. The condensate and gaseous components from condenser pass into decanter 160 through line 150. Decanter 160 may be an overhead receiver for collecting the condensed liquid portion and may have a coalescing member for use in phase separation. In some embodiments, the condensate and gaseous components are directed in separate lines and in other embodiments, both components may be passed together in a shared line. In some embodiments, the condensate in line 150 may be further condensed in one or more optional condensers operating at successively lower temperatures. After each subsequent condensing step, both the condensate and gaseous components pass together until they are separated in decanter 160.

In some embodiments, the average residence time of the condensed components in decanter 160 is greater than or equal to 1 minute, e.g., greater than or equal to 3 minutes, greater than or equal to 5 minutes, greater than or equal to 10 minutes. In some embodiments, the average residence time of the condensed components in decanter 160 is less than or equal to 60 minutes, e.g., less than or equal to 45 minutes, less than or equal to 30 minutes, or less than or equal to 25 minutes. The condensate components introduced into decanter 160 are separated into a light phase 162 (which may be referred to as a light phase) and a heavy phase 164 (which may be referred to as a heavy phase). Light phase 162 primarily includes water typically with methyl acetate, acetic acid, hydrogen iodide, acetaldehyde, dimethyl ether, methanol, and lesser amounts of methyl iodide. Methyl iodide concentrates in heavy phase 164 which further includes methyl acetate, acetaldehyde, dimethyl ether, hydrogen iodide, methanol, and lesser amounts of water and acetic acid. In some embodiments, the acetaldehyde concentration in light phase 162 is larger, based on wt.%, than heavy phase 164. Although the specific compositions of light phase 162 may vary widely, some exemplary compositions are provided below in Table 1.

TABLE 1

Exemplary Light Phase from Light Ends Overhead

| | conc. (Wt.%) | conc. (Wt.%) | conc. (Wt.%) |
|---|---|---|---|
| Water | 40-80 | 50-75 | 70-75 |
| Methyl Acetate | 1-50 | 1-25 | 1-15 |
| Acetic Acid | 1-40 | 1-25 | 5-15 |
| PRC’s (AcH) | <5 | <3 | <1 |
| Methyl Iodide | <10 | <5 | <3 |
| Hydrogen Iodide | < 1 | <0.5 | 0.001-0.5 |

In some embodiments, a portion of light phase 162 may be refluxed through line 163 into first column 140. The reflux ratio (the mass flow rate of the reflux divided by the total mass flow exiting the top of column 140, including both heavy phase 164, which may or may not be fully recycled, and light phase 162) of light phase 162 via line 163 to first column 140 is from 0.05 to 0.4, e.g., from 0.1 to 0.35, or from 0.15 to 0.3. In some embodiments, another portion of light phase 162 may be returned to reactor 100 via line 166.

Heavy phase 164, which is primarily methyl iodide, is recycled into the reactor 100. In some embodiments, a portion of heavy phase 164 may be refluxed with the light phase 162 to the first column 140. The specific gravity of heavy phase 164 may be from 1.3 to 2, e.g., from 1.5 to 1.8, from 1.5 to 1.75 or from 1.55 to 1.7. As described in U.S. Pat. No. 6,677,480, the measured specific gravity in heavy phase 164 may correlate to the methyl acetate concentration in the reaction medium. As specific gravity decreases, the methyl acetate concentration in the reaction medium increases. In some embodiments, overhead decanter 160 is arranged and constructed to maintain a low interface level to prevent an excess hold up of methyl iodide. Although the specific compositions of heavy phase 164 may vary widely, some exemplary compositions are provided below in Table 2.

TABLE 2

Exemplary Heavy phase from Light Ends Overhead

| | conc. (Wt.%) | conc. (Wt.%) | conc. (Wt.%) |
|---|---|---|---|
| Water Methyl Acetate | <3 0.1-25 | 0.05-1 0.5-20 | 0.01-1 0.7-15 |
| Acetic Acid | 0.1-10 | 0.5-10 | 0.7-10 |
| PRC’s (AcH) | <5 | <3 | 0.05-0.5 |
| Methyl Iodide | 60-98 | 60-95 | 80-90 |
| Hydrogen Iodide | < 1 | <0.5 | 0.001-0.5 |

The gaseous components from decanter 160 are vented as needed. The vented gaseous components may include carbon monoxide, carbon dioxide, hydrogen, nitrogen, oxygen, methane, acetic acid, methyl acetate, methanol, water, acetaldehyde, dimethyl ether, methyl iodide, hydrogen iodide, and combinations. A condensed portion of the vented gaseous components may be returned.

### Purification for Acetic Acid Product

Turning to light ends column 140 show in FIG. 1, where a side stream 144 withdraw above the feed location. The side stream 144 may comprise acetic acid in an amount from 90 to 99.5 wt.%, water in an amount from 1 to 3 wt.%, methyl iodide in an amount from 0.1 to 5 wt.%, and methyl acetate in an amount from 0.1 to 5 wt.%. As described herein, acetic acid removed via side stream 144 is preferably subjected to further purification as shown in FIG. 1, such as in a second column 180, also referred to as a dehydrating or drying column. A subsequent columns (not shown) may be used in some embodiments to further remove high-boiling point impurities that include but are not limited to propionic acid or acetic anhydride. In some embodiments, a portion of side stream 144 may be condensed and fed back into first column 140. The concentration of catalyst in side stream 144 is typically about one or two order of magnitudes lower than the catalyst concentration in vapor stream 122 coming off flash vessel 120, but side stream 144 may still contain low levels of catalyst. In further embodiments, to remove any metal entrainment in side stream 144, a portion of the side stream may be treated in a fixed bed containing a polymer having nitrogen-containing heterocyclic repeat units, such as pyridine or pyrrolidone polymer, to sequester catalyst prior to entering second column 180, as described in U.S. Pat. No. 7,902,398.

In some embodiments, second column 180 may include a combination of different distillation apparatuses. For example, a combination of bubble-cap column and perforated plate column may be used as well as a combination of perforated plate column and a packed column. In some embodiments, second column 180 may be a plate column having up to 120 theoretical plates, e.g., from 2 to 120, from 5 to 80, from 5 to 60 theoretical plates. The column top gauge pressure may be from 2 atm to 4 atm with a temperature from 130° C. to 155° C. and a higher bottom gauge pressure from 2.5 atm to 5 atm with a temperature from 150° C. to 180° C.

Second column 180 separates side stream 144 to form overhead stream 182 comprised primarily of water and product stream 184 comprised primarily of acetic acid, which may be removed at base or near base, as shown by stream 184, of second column 180. When removed near the base stream 184 may be taken within 5 trays of the base. Overhead stream 182 may comprise water in an amount from 50 to 95 wt.%, e.g., from 50 to 90 wt.%, or from 50 to 75 wt.%. Methyl acetate and methyl iodide are also removed from side stream 144 and concentrated in overhead stream 182. Overhead stream 182 may be condensed and refluxed as needed to the second column, with the remainder of the overhead stream being returned to reactor 100. To prevent excess iodide build up in second column 180, a decanter may phase separate the condensed overhead stream 182 and an aqueous portion is used as the reflux.

One useful function of second column 180 is to limit the iodides in product stream 184. The iodides, if not removed up to this point, are further removed out by guard beds 198 and it is desirable to operate with low iodide content to maintain a useful lifetime for the guard beds. In some embodiments, to further limit the iodides in the product stream, at least one substance selected from the group consisting of methanol, methyl acetate, and potassium hydroxide may be fed to second column 180 or the side stream fed to second column 180. These substances are known to react particularly favorably with corrosive iodides, such as hydrogen iodides, so that the iodides can be favorably be removed in the overhead and returned to the reactor.

### Guard Beds

A low total iodide concentration, e.g., up to 5 wppm, e.g., up to 1 wppm, is needed for the feed to the guard bed. The use of one or more guard beds to remove residual iodide greatly improves the quality of the purified acetic acid product. Carboxylic acid streams, e.g., acetic acid streams, that are contaminated with halides and/or corrosion metals may be contacted with the ion exchange resin composition under a wide range of operating conditions. Preferably, the ion exchange resin composition is provided in a guard bed. The use of guard beds to purify contaminated carboxylic acid streams is well documented in the art, for example, U.S. Pat. Nos. 4,615,806; 5,653,853; 5,731,252; and 6,225,498, which are hereby incorporated by reference in their entireties. Generally, a contaminated liquid carboxylic acid stream is contacted with the ion exchange resin composition, which is preferably disposed in the guard bed. The halide contaminants, e.g., iodide contaminants, react with the metal to form metal iodides. In some embodiments, hydrocarbon moieties, e.g., methyl groups, that may be associated with the iodide may esterify the carboxylic acid. For example, in the case of acetic acid contaminated with methyl iodide, methyl acetate would be produced as a byproduct of the iodide removal. The formation of this esterification product typically does not have a deleterious effect on the treated carboxylic acid stream.

In some embodiments, the ion exchange resin is a metal-exchanged ion exchange resin and may comprise at least one metal selected from the group consisting of silver, mercury, palladium and rhodium. In some embodiments, at least 1% of the strong acid exchange sites of said metal-exchanged resin are occupied by silver. In another embodiment, at least 1% of the strong acid exchange sites of said metal-exchanged resin are occupied by mercury. The process may further comprise treating the purified acetic acid product with a cationic exchange resin to recover any silver, mercury, palladium or rhodium.

The pressure during the contacting step is limited primarily by the physical strength of the resin. In some embodiments, the contacting is conducted at pressures ranging from 1 to 10 atm, e.g., from 1 to 8 atm, or from 1 to 5 atm. For convenience, however, both pressure and temperature preferably may be established so that the contaminated carboxylic acid stream is processed as a liquid. Thus, for example, when operating at atmospheric pressure, which is generally preferred based on economic considerations, the temperature may range from 17° C. (the freezing point of acetic acid) to 118° C. (the boiling point of acetic acid). It is within the purview of those skilled in the art to determine analogous ranges for product streams comprising other carboxylic acid compounds. The temperature of the contacting step preferably is kept relatively low to minimize resin degradation. In some embodiments, the contacting is conducted at a temperature ranging from 25° C. to 120° C., e.g., from 25° C. to 100° C., or from 50° C. to 100° C. Some cationic macroreticular resins typically begin degrading (via the mechanism of acid-catalyzed aromatic desulfonation) at temperatures of 150° C. Carboxylic acids having up to 5 carbon atoms, e.g., up to 3 carbon atoms, remain liquid at these temperatures. Thus, the temperature during the contacting should be maintained below the degradation temperature of the resin utilized. In some embodiments, the operating temperature is kept below temperature limit of the resin, consistent with liquid phase operation and the desired kinetics for halide removal.

The configuration of the guard bed within an acetic acid purification train may vary widely. For example, the guard bed may be configured after a drying column. Additionally or alternatively, the guard be may be configured after a heavy ends removal column or finishing column. Preferably, the guard bed is configured in a position where the temperature acetic acid product stream is low, e.g., less than or equal to 120° C., or less than or equal to 100° C. Aside from the advantages discussed above, lower temperature operation provides for less corrosion as compared to higher temperature operation. Lower temperature operation provides for less formation of corrosion metal contaminants, which, as discussed above, may decrease overall resin life. Also, because lower operating temperatures result in less corrosion, vessels advantageously need not be made from expensive corrosion-resistant metals, and lower grade metals, e.g., standard stainless steel, may be used.

In some embodiments, the flow rate through the guard bed ranges from 0.1 bed volumes per hour (“BV/hr”) to 50 BV/hr, e.g., 1 BV/hr to 20 BV/hr or from 6 BV/hr to 10 BV/hr. A bed volume of organic medium is a volume of the medium equal to the volume occupied by the resin bed. A flow rate of 1 BV/hr means that a quantity of organic liquid equal to the volume occupied by the resin bed passes through the resin bed in a one hour time period.

To avoid exhausting the resin with a purified acetic acid product that is high in total iodide concentration, in some embodiments the purified acetic acid product in bottoms stream 184 is contacted with a guard bed when total iodide concentration of the purified acetic acid product is up to 5 wppm, e.g., preferably up to 1 wppm. In one exemplary embodiment, the total iodide concentration of the purified acetic acid product may be from 0.01 wppm to 5 wppm, e.g., from 0.01 wppm to 1 wppm. Concentrations of iodide above 5 wppm may require re-processing the off-spec acetic acid. Total iodide concentration includes iodide from both organic, C₁ to C₁₄ alkyl iodides, and inorganic sources, such as hydrogen iodide. A purified acetic acid composition is obtained as a result of the guard bed treatment. The purified acetic acid composition, in some embodiments, comprises less than 100 wppb iodides, e.g., less than 90 wppb, less than 50 wppb, or less than 25 wppb. In some embodiments, the purified acetic acid composition comprises less than 1000 wppb corrosion metals, e.g., less than 750 wppb, less than 500 wppb, or less than 250 wppb. For purposes of the present invention, corrosion metals include metals selected from the group consisting of nickel, iron, chromium, molybdenum and combinations thereof. In terms of ranges, the purified acetic acid composition may comprise from 0 to 100 wppb iodides, e.g., from 1 to 50 wppb; and/or from 0 to 1000 wppb corrosion metals, e.g., from 1 to 500 wppb. In other embodiments, the guard bed removes at least 25 wt.% of the iodides from the crude acetic acid product, e.g., at least 50 wt.%, or at least 75 wt.%. In some embodiments, the guard bed removes at least 25 wt.% of the corrosion metals from the crude acetic acid product, e.g., at least 50 wt.%, or at least 75 wt.%.

In another embodiment, the product stream may be contacted with cationic exchanger to remove lithium compounds. The cationic exchanger in the acid form comprises a resin of acid-form strong acid cation exchange macroreticular, macroporous or mesoporous resins. Without being bound by theory, feeding a product stream to an ion-exchange comprising lithium compounds in an amount of greater than or equal to 10 wppm results in displacement of metals in the treated product. Advantageously, this may be overcome by using a cationic exchanger upstream of the ion-exchange resin. After contacting with the cationic exchanger, the product stream may have a lithium ion concentration of less than or equal to 50 weight part per billion (wppb), e.g., less than or equal to 10 wppb, or less than or equal to 5 wppb.

Although the product stream may be contacted with an ion-exchange resin to remove iodides, it is preferred not to flash the product stream or contact with product stream with an adsorption system that contains activated carbon. Flashing the product stream is not efficient because there is not a sufficient pressure drop to recover greater than 50% of the acetic acid from the product stream. Thus, in some embodiments, a non-flashed portion of the product stream is fed to the ion-exchange bed to remove iodides.

### Acetaldehyde Removal

Turning now to the removal and/or reduction of permanganate reducing components (PRC), which includes acetaldehyde among other components. FIG. 1 indicates that a portion of light phase 162′ and/or heavy phase 164′ may be directed to a PRC removal system 200. Either portion may be selected depending on the processing requirements. In general the process for removing and reducing PRC involves removing water from either stream to obtain a process stream and then combining the process stream with at least one alkane, such as butane and/or pentane. For the purposes of the present disclosure the alkane does not include more than trace amounts of hydrocarbons having from six to twenty carbons, in particular decane and dodecane. In trace amounts such hydrocarbons are not in sufficient quantity to effect separation, e.g., in amounts of less than 0.001 wt.%. In one embodiment, the alkane comprises from 60 to 100% of butane and/or pentane, e.g., from 75 to 99.9% or from 80 to 99.5%. In addition to linear forms, there also may be branched isomers of butane and/or pentane. The use of an alkane functions to improve the separation of PRCs and in particular the separation of PRCs from alkyl iodides, such as methyl iodide, into a purge stream. Without being bound by theory, the alkane is added to a stream with low amounts of water and may cause formation of one or more azeotropes.

To illustrate the various embodiments, the removal and/or reduction of acetaldehyde from light phase 162′ is further shown in FIG. 2, and the removal and/or reduction of acetaldehyde from heavy phase 164′ is further shown in FIG. 3. It should be understood that in each of these embodiments that a combination of light phase 162′ and heavy phase 164′ may be processed together in either process. Other variations are within the scope to first remove water and then combine with an alkane.

In FIG. 2, a portion of the light phase, shown by stream 162′, is fed to a distillation column 202. Distillation column 202 may be a plate column, a packed column, or other column having a sufficient number of theoretical stages to remove not less than 80% of the water from light phase 162′, e.g. not less than 90% or not less than 95%. In one embodiment, theoretical stages in distillation column may be from 2 to 100, e.g., from 5 to 80. The light phase 162′ may be fed at a suitable location that is below the optional sidedraws (if used) or if no sidedraws below the top tray of the distillation column 202. To avoid the alkane from being returned to the reactor, it is desirable to avoid contacting the light phase 162′ directly with an alkane.

In one embodiment, distillation column 202 operates under a temperature from 50° C. to 180° C., e.g., from 70° C. to 140° C., and a pressure from 50 to 500 kPa, e.g., 100 kPa to 300 kPa, to yield a bottom stream 204 that contains a majority of the water from the light phase 162′. Bottom stream 204 may be returned to the reactor 100 or other suitable location in the process to maintain a suitable water concentration. This first stage separation provides an overhead 206 that is suitable for combining with an alkane because it is deficient in water. Overhead 206 contains an enriched portion of the PRCs as well as alkyl iodides.

In one embodiment, there may be a sidedraw 208 taken from distillation column 202. A sidedraw may be used to further reduce the methyl acetate concentration in the overhead 206.

Overhead 206 is condensed by one or more condensers and reflux to distillation column at a reflux ratio (the mass flow rate of the reflux divided by the total mass flow exiting the top of column 202) from 0.05 to 0.8, e.g., from 0.05 to 0.4 or from 0.1 to 0.3. The uncondensed portion in stream 210 is collected in receiver 212 to return a liquid portion 214 that is combined with the condensed portion from stream 216. In doing so the PRCs in stream 210 may be removed from the vent.

In one embodiment, liquid portion 214 that is combined with the condensed portion from stream 216 in a mixer 218, which may be an in-line mixer, a static mixer, hold tank or other suitable vessel. As a result of the mixing, the combined process stream 220 has a water concentration of no more than 1.5 wt.%, e.g., no more than 1.2 wt.% or no more than 1.0 wt.%. In some embodiments, the process stream may be formed by combined liquid portion 214 and stream 216 without mixer.

The combined process stream 220 has a water concentration of no more than 1.5 wt.%, e.g., no more than 1.2 wt.%, or no more than 1.0 wt.%. The combined process stream 220 is further directed to primary vessel 230 and is combined with an alkane stream 232 comprising an alkane (e.g., butane). Although, the alkane stream 232 is shown being fed into the vessel 230 in other embodiments, alkane stream 232 may be combined in vessel 218 or into combined process stream 220.

In FIG. 2, the primary vessel 230 is depicted as a decanter, but in other embodiments the primary vessel 230 may be an extractor, mixer, hold tank, or similar vessel. Although two phases may be maintained in primary vessel 230, the PRC components favorably distribute into the organic phase 234 over the aqueous phase 236. Because of the low water content the aqueous phase 236 is a relatively small stream and can be discarded or recycled as needed. The alkanes and PRCs, along with alkyl iodides, are withdrawn through the organic phase 234, which is further directed to a distillation column 240. The organic phase 234, which may also be referred to as a combined stream, has a composition of alkanes from 10 wt.% to 55 wt.%, e.g., from 15 wt.% to 45 wt.%, PRCs from 5 wt.% to 45 wt.%, e.g., from 10 wt.% to 40 wt.%, and alkyl iodides from 10 wt.% to 75 wt.%, e.g., from 15 wt.% to 70 wt.%.

Distillation column 240 is effective separating PRCs from alkyl iodides, e.g., methyl iodide, through distillation. Prior methods to remove alkyl iodides have found little success in separating these components through distillation, without using an extractive agent. The overhead 242 contains the PRCs, acetaldehyde, and alkanes, while the lower stream 244 contains an enriched portion of the alkyl iodides. In terms of concentration, the lower stream 244 contains no less than 60 wt.% of methyl iodide, e.g., no less than 65 wt.% of methyl iodide or no less than 70 wt.% of methyl iodide. In one embodiment, the lower stream 244 contains substantially no amount of butane and thus, the lower stream 244 may be returned to the reactor 100 or other suitable location in the process without introducing butane to the reactor.

Although the overhead 242 has removed PRCs from the process it may be desirable to further recover alkyl iodides. This may be done through water extraction as taught by U.S. Pat. Nos. 7,855,306; 7,223,886; and 6,143,930, the entire contents and disclosure which are incorporated by reference. FIG. 2 illustrates one embodiment for recovering alkyl iodides. Overhead 242 is condensed by one or more condensers, the condensed portion 246 is refluxed as needed and both the condensed portion 246 and vapor portion 248 is collected in secondary vessel 250. In FIG. 2, secondary vessel 250 is depicted as a decanter, but in other embodiments secondary vessel may be an extractor, mixer, or similar vessel. A water stream 252 may be introduced into secondary vessel 250 to enhance phase separation. In one embodiment, water stream 252 may contain from 60 wt.% to 100 wt.% water, e.g., from 70 wt.% to 99 wt.% water or from 80 to 98.5 wt.% water. The PRC and added water are phased in the aqueous phase 254 and may be purged from the system. In one embodiment, the purged aqueous phase 254 contains low amounts of methyl iodide of not more than 1.5 wt.%, e.g., not more than 1.0 wt.%, or not more than 0.4 wt.%. The butane is separated in the organic phase 256 and may be used as the alkane stream 232 or directed elsewhere.

Turning now to the processing of the heavy phase 164′ in FIG. 3, the PRC removal system 200 operates with distillation column 302 and the heavy phase 164′ has less water than the aqueous phase 162′. Distillation column 302 may be a plate column, a packed column, or other column. In one embodiment, theoretical stages in distillation column may be from 2 to 100, e.g., from 5 to 80. The heavy phase 164′ may be fed at a suitable location that is below mid-point of the column 302 or near the base of column 302, e.g., within 5 trays of the base. In one embodiment, distillation column 302 operates under a temperature from 50° C. to 180° C., e.g., from 70° C. to 140° C., and a pressure from 50 to 500 kPa, e.g., 100 kPa to 300 kPa, to yield a bottom stream 304 that contains a majority of the methyl iodide. Although the relative amount of water is lower in the heavy phase 164′ compared with the light phase 162′, the water is reduced by operation of distillation column 302 that removes from 80 to 99% of the heavy phase 164′ into the bottom 304 and 1 to 10% of the heavy phase 164′ into the overhead 306. As indicated above a portion of the aqueous phase 162′ may be combined with the heavy phase 164′, which increase the water content.

To avoid the alkane from being returned to the reactor, it is desirable to avoid contacting the heavy phase 164′ directly with an alkane prior to being introduced to distillation column 302.

The overhead 306 is condensed by one or one condensers and collected in a receiver 308. Receiver 308 operates under conditions to dehydrate or reduce the water content of the overhead 306. Venting may be used as needed. In one embodiment, receiver 308 may be a phase separation vessel such as a decanter. In such embodiments, an aqueous stream 310 may be withdrawn and discarded or retained by recycling. The organic phase 312 is refluxed and a portion, which is a process stream, is introduced to a primary vessel 320. The organic phase 312 has a water concentration of no more than 0.9 wt.%, e.g., no more than 0.6 wt.%, or no more than 0.4 wt.%. Thus, water is removed to obtain from the overhead to obtain a process stream suitable for further processing.

In FIG. 3, the primary vessel 320 is depicted as a decanter, but in other embodiments the primary vessel 320 may be an extractor, mixer, or similar vessel. Although two phases may be maintained in primary vessel 320, the PRC components favorably distribute, along with butane, into the organic phase 324 over the aqueous phase 326. Because the aqueous phase 326 is a relatively small stream, it can be discarded or recycled as needed. The alkanes and PRCs, along with alkyl iodides, are withdrawn through the organic phase 324 as a combined stream, which is further directed to a distillation column 330. The organic phase 324, which may also be referred to as a combined stream, has a composition of alkanes from 5 wt.% to 40 wt.%, e.g., from 10 wt.% to 35 wt.%, PRCs from 1 wt.% to 30 wt.%, e.g., from 2 wt.% to 25 wt.%, and alkyl iodides from 40 wt.% to 80 wt.%, e.g., from 45 wt.% to 75 wt.%.

Distillation column 330 is effective separating PRCs from alkyl iodides, e.g., methyl iodide, through distillation. In one embodiment, distillation column 330 operates under a temperature from 50° C. to 180° C., e.g., from 65° C. to 120° C., and a pressure from 50 to 500 kPa, e.g., 150 kPa to 350 kPa. Prior methods to remove alkyl iodides have found little success in separating these components through distillation, without using an extractive agent or other costly distillation processes. The overhead 332 contains the PRCs, acetaldehyde, and alkanes, while the lower stream 334 contain an enriched portion of the alkyl iodides, e.g., methyl iodide. In one embodiment, lower stream 334 has a composition of alkyl iodide that is not less than 60 wt.%, e.g., not less than 65 wt.%, or not less than 70 wt.%. The lower stream 334 may be combined with the bottoms 304 and returned to the reactor 100 or other suitable location in the process.

Although the overhead 332 has removed PRCs from the process it may be desirable to further recover alkyl iodides, as described above by using known water extraction. Like FIG. 2, FIG. 3 illustrates one embodiment for recovering alkyl iodides. Overhead 332 is condensed by one or more condensers, the condensed portion 336 is refluxed as needed and both the condensed portion 336 and vapor portion 338 are fed and collected in secondary vessel 340. In some embodiments, the condensed portion 336 and vapor portion 338 are fed in separate lines. In FIG. 3, secondary vessel 340 is depicted as a decanter, but in other embodiments the secondary vessel may be an extractor, mixer, or similar vessel. In one embodiment, the overhead is biphasically separated. A water stream 342 may be introduced into secondary vessel 340 to enhance phase separation. In one embodiment, water stream 342 may contain from 60 wt.% to 100 wt.% water, e.g., from 70 wt.% to 99 wt.% water or from 80 to 98.5 wt.% water. The PRC and added water are phased in the aqueous stream 344 and may be purged from the system. Thus, in one embodiment, the loss of methyl iodide may be reduced in the aqueous stream 344 to provide a mass ratio of methyl iodide to acetaldehyde being less than or equal to 0.1, e.g., less than or equal to 0.05 or less than or equal to 0.03. The butane is separated in the organic stream 346 and may be used as the alkane stream 322.

The distillation columns described herein may be conventional distillation columns, e.g., a plate column, a packed column, and others, and combinations thereof. Plate columns may include a perforated plate column, bubble-cap column, Kittel tray column, uniflux tray, or a ripple tray column. For a plate column, the theoretical number of plates is not particularly limited and depending on the species of the component to be separated, may include up to 80 plates, e.g., from 2 to 80, from 5 to 60, from 5 to 50, or more preferably from 7 to 35. The distillation column may include a combination of different distillation apparatuses. Unless excluded, a combination of bubble-cap column and perforated plate column may be used, as well as a combination of perforated plate column and a packed column.

The material of each member or unit associated with the distillation system, including the columns, valves, condensers, receivers, pumps, reboilers, and internals, and various lines, each communicating to the distillation system may be made of suitable materials such as glass, metal, ceramic, or combinations thereof, and is not particularly limited to a specific one. According to the present invention, the material of the foregoing distillation system and various lines are a transition metal or a transition-metal-based alloy such as iron alloy, e.g., a stainless steel, nickel or nickel alloy, zirconium or zirconium alloy thereof, titanium or titanium alloy thereof, or aluminum alloy. Suitable iron-based alloys include those containing iron as a main component, e.g., a stainless steel that also comprises chromium, nickel, molybdenum and others. Suitable nickel-based alloys include those containing nickel as a main component and one or more of chromium, iron, cobalt, molybdenum, tungsten, manganese, and others, e.g., HASTELLOY™ and INCONEL™. Corrosion-resistant metals may be particularly suitable as materials for the distillation system and various lines.

At the outset, it should be noted that in the development of any such actual embodiment, numerous implementation-specific decisions must be made to achieve the developer’s specific goals, such as compliance with system related and business related constraints, which will vary from one implementation to another. In addition, the processes disclosed herein can also comprise components other than those cited or specifically referred to, as is apparent to one having average or reasonable skill in the art.

As is evident from the figures and text presented above, a variety of embodiments are contemplated.

The present invention will be better understood in view of the following non-limiting examples.

### EMBODIMENTS

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a method for removing permanganate reducing compounds (PRCs) formed in the production of acetic acid, comprising: reacting methanol and carbon monoxide in a suitable liquid phase reaction medium comprising a Group VIII metal catalyst, an alkyl iodide, and water; separating the liquid phase reaction medium into a vapor stream comprising acetic acid and at least one PRCs, and a residuum catalyst stream; distilling at least a portion of the vapor stream in a first distillation column to yield a side stream comprising acetic acid and a first overhead comprising alkyl iodide, water, and PRCs; removing water from at least a portion of the first overhead to obtain a process stream, combining at least a portion of the process stream with at least one alkane to obtain a combined stream; distilling the combined stream to yield a second overhead comprising the at least one alkane and PRCs, and a lower stream comprising the alkyl iodide; and biphasically separating the second overhead to remove PRCs in an aqueous stream.

Embodiment 2 is the method of embodiment(s) 1, wherein the at least one alkane comprises butane.

Embodiment 3 is the method of embodiment(s) 1, wherein the PRCs comprise acetaldehyde, acetone, methyl ethyl ketone, butylaldehyde, crotonaldehyde, 2-ethyl crotonaldehyde, 2-ethyl butyraldehyde, or the aldol condensation products thereof, including mixtures thereof.

Embodiment 4 is the method of embodiment(s) 1, wherein the process stream contains water at a concentration of no more than 1.5 wt.%.

Embodiment 5 is the method of embodiment(s) 1, wherein the process stream is combined with the at least one alkane in a primary vessel, wherein the primary vessel may be a decanter, extractor, mixer or hold tank.

Embodiment 6 is the method of embodiment(s) 5, wherein the primary vessel is a decanter and the combined stream is withdrawn as an organic phase.

Embodiment 7 is the method of embodiment(s) 1, wherein the combined stream comprises the at least one alkane, alkyl iodide and at least one PRCs.

Embodiment 8 is the method of embodiment(s) 1, wherein the water is removed from the at least a portion of the first overhead in a distillation column.

Embodiment 9 is the method of embodiment(s) 8, wherein the water is further removed in a decanter.

Embodiment 10 is the method of embodiment(s) 1, wherein the second overhead is biphasically separated in a secondary vessel, wherein the secondary vessel may be a decanter, extractor, mixer or hold tank.

Embodiment 11 is the method of embodiment(s) 10, further comprising adding a water stream comprising from 60 wt.% to 100 wt.% of water to the secondary vessel.

Embodiment 12 is the method of embodiment(s) 10, wherein the aqueous stream has a mass ratio of methyl iodide to acetaldehyde of less than or equal to 0.1.

Embodiment 13 is the method of embodiment(s) 10, further comprising separating an organic stream comprising the alkyl iodide from the secondary vessel.

Embodiment 14 is the method of embodiment(s) 10, wherein the aqueous stream comprises methyl iodide concentrations of not more than 1.5 wt.%.

Embodiment 15 is the method of embodiment(s) 1, wherein the first overhead is separated into a light phase comprising water and a heavy phase comprising the alkyl iodide.

### EXAMPLES

### Example 1

A simulation using computer modelling was conducted on a reproduced heavy phase having a composition of that representative of the heavy phase obtained from the decanter of a light ends column. The reproduced heavy phase was processed according to FIG. 3. The composition of the reproduced stream was 17.3% by mass of methyl acetate, 1.9% by mass acetic acid, 1% by mass of water, 0.2% by mass of methanol and 2550 ppm of acetaldehyde, with the balance being methyl iodide. 100 parts of the reproduced heavy phase was fed to a distillation column operating with a base temperature of 68.9° C., and a column pressure of 241 kPa. 98.7 parts of the reproduced heavy phase was withdrawn from the bottom of the column, while the remaining 1.3 parts was carried overhead. The reflux ratio of refluxed portion to overhead was 0.96. The bottom contained less acetaldehyde than the reproduced heavy phase. The mass ratio of water in the overhead to the bottom was 0.016, which indicates a significant portion of the water was removed by the distillation column. The overhead composition contained 16.79% by mass of acetaldehyde, 1.2% by mass of water, 1.2% by mass methyl acetate.

After being condensed the overhead was collected in a decanter and a small aqueous stream was removed to further reduce the water concentration. The organic phase was used as the reflux and the remaining portion was fed to a primary vessel along with a stream comprising 78.7% by mass of butane. Additional water was removed in an aqueous phase, leaving an organic phase. The composition of the organic phase was 69.3% by mass of methyl iodide, 15.6% by mass of acetaldehyde, 12.4% by mass of butane, 0.88% by mass of water, and the balancing being methyl acetate, methanol and acetic acid. This organic phase was fed to a distillation column operating with a base temperature of 64.7° C. and a column pressure of 310 kPa. The butane was distilled into the overhead along with a portion of the acetaldehyde and some methyl iodide. The overhead was further condensed and collected in a secondary vessel in which water was added. The acetaldehyde was purged from the process in an aqueous stream.

To demonstrate the benefit of adding butane to a stream having a low content of water, a comparison was run with no butane addition. When no butane addition was used, the ratio of methyl iodide to acetaldehyde in the purged stream was significant higher. This demonstrates that the butane addition lead to lower methyl iodide losses and an improved process.

TABLE 3

| | Comparative Example | Inventive Example 1 |
|---|---|---|
| Butane Added | No | Yes |
| Aqueous Stream | | |
| Acetaldehyde | 24.65% by mass | 25.51% by mass |
| Methyl Iodide | 3.97% by mass | 0.52% by mass |
| Ratio | 0.16 | 0.02 |

### Example 2

A simulation using computer modelling was conducted on a reproduced light phase having a composition of that representative of the light phase obtained from the decanter of a light ends column. The reproduced light phase was processed according to FIG. 2. The composition of the reproduced light phase was 69.1% by mass of water, 20.1% by mass acetic acid, 7.9% by mass of methyl acetate, 0.85% by mass of methanol, 0.7% by mass of methyl iodide and 3962 ppm of acetaldehyde.

100 parts of the reproduced light phase was fed to a distillation column operating with a base temperature of 109.7° C., and a column pressure of 241 kPa. 97.7 parts of the reproduced heavy phase was withdrawn from the bottom of the column, while the remaining 2.3 parts was carried overhead. The bottom composition was 70.7% by mass of water, 20.5% by mass acetic acid, 7.9% by mass of methyl acetate, 0.87% by mass of methanol, and less than 200 ppm of acetaldehyde.

The overhead was condensed and the vapor portion was further condensed and separated to return a liquid stream that was combined with the overhead to form a mixed stream. The mixed stream composition was 53.3% by mass of methyl iodide and 28.9% by mass of acetaldehyde, 0.97% by mass of water, 16.35% by mass of methyl acetate, and the balance being comprised of methanol and acetic acid. The mixed stream was fed to a primary vessel along with a stream comprising butane. The butane mixed with the mixed stream and formed two phases, a relatively small aqueous stream and an organic stream containing the butane and acetaldehyde. The organic stream was further distilled in a distillation column, in which the butane was carried overhead. After being condensed the overhead was contacted with a water stream in a secondary vessel. A purge stream comprising 75.4% by mass water, 23.4% by mass acetaldehyde and 0.24% by methyl iodide was removed from the process. Butane was also recovered from the secondary vessel.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

1. A method for removing permanganate reducing compounds (PRCs) formed in the production of acetic acid, comprising:
reacting methanol and carbon monoxide in a liquid phase reaction medium comprising a Group VIII metal catalyst, an alkyl iodide, and water;
separating the liquid phase reaction medium into a vapor stream comprising acetic acid and at least one PRCs, and a residuum catalyst stream;
distilling at least a portion of the vapor stream in a first distillation column to yield a side stream comprising acetic acid and a first overhead comprising alkyl iodide, water, and PRCs;
removing water from at least a portion of the first overhead to obtain a process stream,
combining at least a portion of the process stream with at least one alkane to obtain a combined stream;
distilling the combined stream to yield a second overhead comprising the at least one alkane and PRCs, and a lower stream comprising the alkyl iodide; and
biphasically separating the second overhead to remove PRCs in an aqueous stream.

2. The method of claim 1, wherein the at least one alkane comprises butane.

3. The method of claim 1, wherein the PRCs comprise acetaldehyde, acetone, methyl ethyl ketone, butylaldehyde, crotonaldehyde, 2-ethyl crotonaldehyde, 2-ethyl butyraldehyde, or aldol condensation products thereof, including mixtures thereof.

4. The method of claim 1, wherein the process stream contains water at a concentration of no more than 1.5 wt.%.

5. The method of claim 1, wherein the process stream is combined with the at least one alkane in a primary vessel, wherein the primary vessel may be a decanter, extractor, mixer or hold tank.

6. The method of claim 5, wherein the primary vessel is a decanter and the combined stream is withdrawn as an organic phase.

7. The method of claim 1, wherein the combined stream comprises the at least one alkane, alkyl iodide and at least one PRCs.

8. The method of claim 1, wherein the water is removed from the at least a portion of the first overhead in a distillation column.

9. The method of claim 8, wherein the water is further removed in a decanter.

10. The method of claim 1, wherein the second overhead is biphasically separated in a secondary vessel, wherein the secondary vessel may be a decanter, extractor, mixer or hold tank.

11. The method of claim 10, further comprising adding a water stream comprising from 60 wt.% to 100 wt.% of water to the secondary vessel.

12. The method of claim 10, wherein the aqueous stream has a mass ratio of methyl iodide to acetaldehyde of less than or equal to 0.1.

13. The method of claim 10, further comprising separating an organic stream comprising the alkyl iodide from the secondary vessel.

14. The method of claim 10, wherein the aqueous stream comprises methyl iodide concentrations of not more than 1.5 wt.%.

15. The method of claim 1, wherein the first overhead is separated into a light phase comprising water and a heavy phase comprising the alkyl iodide.
